(19) United States
(12) Patent Application Publication
(10) Pub. No.: US 2025/0114122 A1
(43) Pub. Date: Apr. 10, 2025

(54) SURGICAL VISUALIZATION ASSEMBLY AND SYSTEM

(71) Applicant: Relign Corporation, Campbell, CA (US)

(72) Inventor: Aaron Germain, San Jose, CA (US)

(21) Appl. No.: 18/828,634

(22) Filed: Sep. 9, 2024

Related U.S. Application Data
(60) Provisional application No. 63/588,382, filed on Oct. 6, 2023.

(51) Int. Cl. A61B 17/34; A61B 17/00; A61B 90/00

(52) U.S. Cl. CPC A61B 17/3423; A61B 90/361; A61B 2017/00084; A61B 2017/00115; A61B 2017/00137; A61B 2017/00199; A61B 2017/00477; A61B 2017/00862

(57) ABSTRACT

A cannula configured to provide an access port to a patient for an arthroscopic or laparoscopic device, optionally comprising: an elongate shaft having a proximal end portion and a distal end portion, wherein the elongate shaft includes, one or more lumens therein including at least one lumen configured to provide a working channel for the arthroscopic or laparoscopic device to pass through the distal end portion; a visualization assembly including one or more cameras, the visualization assembly coupled to the elongate shaft and having pathway for optical and electrical connection to the one or more cameras with the pathway formed along at least a portion of the elongate shaft.

### CLAIM OF PRIORITY

This application claims the benefit of U.S. Provisional Patent Application Ser. No. 63/588,382, filed on Oct. 6, 2023, the benefit of priority of which is claimed hereby, and which is incorporated by reference herein in its entirety.

### FIELD OF THE INVENTION

The present invention relates generally to apparatuses for imaging during surgical procedures such as an arthroplasty procedure and, more particularly, to cannulas used to visualize the relevant anatomy during treatment of tissue.

### BACKGROUND

Arthroscopic surgery is a minimally invasive procedure performed through small incisions. Such surgeries can use an arthroscope to visualize the joint space. Current cannulas provide channels for inserting surgical instruments into the joint while minimizing fluid leakage. Arthroscopes can be used in concert with a variety of surgical apparatuses including those for endoscopic cutting and removal of bone. Currently, surgeons use shavers and burrs having rotational cutting surfaces to remove hard and soft tissue in such procedures.

Many arthroplasty procedures including those on the hip and shoulder require precise tool positioning due to the challenging anatomy of the hip and shoulder joints. This anatomy can also present visualization challenges as the joints have limited vantage points for visualization. These procedures typically require additional incisions for arthroscope portals.

### Overview

There is a need for an arthroscopic visualization instrument that has integrated imaging capabilities but that can also act as a cannula for access to anatomy for surgical instruments that perform therapy. Additionally, there is a need for an arthroscopic visualization instrument that can meet the imaging needs for the challenging anatomy of the hip, shoulder and other joints. The present inventor proposes an arthroscopic cannula with an integrated camera(s) chip and LED light source(s) such as at the distal tip. This provides direct visualization from the cannula without needing a separate arthroscope. Optionally, the distal tip can be articulated (bent, rotated, translated, etc.) to provide a wider or different viewing angle of the surgical site.

The present inventor has developed a system of using multiple cannulas inserted into the joint space to provide different vantage points that can be merged into a panoramic “amphitheater” view of the therapeutic instrument and/or the entire joint. This can allow for more effective real-time feedback for surgeons. Such multi-instrument visualization can provide for enhanced capabilities during surgery.

The present inventor has also developed an arthroscopic cannula with an integrated infrared (IR) camera chip that can be configured to monitor temperature and detect bleeding. This allows detection of overheating from surgical devices and/or early bleeding before it obscures the visual field.

Optionally, the arthroscopic cannulas discussed can be disposable (designed for single use). The arthroscopic cannulas can provide improved visualization and simplicity compared to standard arthroscopic equipment. Surgeons can gain wider environmental awareness intra-operatively without additional incisions that would otherwise be created only for imaging devices. The instruments disclosed herein can be configured to be reusable as well. Thus, examples provided herein (e.g., reusable v. disposable) are not intended as limiting but are merely provided for exemplary purposes. The present inventor contemplates the apparatuses and systems can reduce costs by eliminating some dedicated imaging tools, reduce surgical complexity particularly in regards to hip and shoulder procedures and improve surgical efficiency among other benefits.

Various relevant arthroplasty and arthroscopic instruments are commonly owned by the applicant. These instruments can benefit from use of the arthroscopic cannulas discussed herein. Relevant patent publications include: U.S. Pat. Nos. 11,065,023; 11,172,953; US 2018-0303509; US 2019-0008541; US 2019-0059983; US 2019-0134279; US 2019-0021788; US 2018-0317957; US 2019-0008538; US 2019-0083121; US 2018-0263649; US 2017-0290602 and US 2019-0015151, the full disclosures of each of which are incorporated herein by reference.

The following, non-limiting examples, detail certain aspects of the present subject matter to solve the challenges and provide the benefits discussed herein, among others.

Example 1 is a cannula configured to provide an access port to a patient for an arthroscopic or laparoscopic device, optionally comprising: an elongate shaft having a proximal end portion and a distal end portion, wherein the elongate shaft includes, one or more lumens therein including at least one lumen configured to provide a working channel for the arthroscopic or laparoscopic device to pass through the distal end portion; a visualization assembly including one or more cameras, the visualization assembly coupled to the elongate shaft and having pathway for optical and electrical connection to the one or more cameras with the pathway formed along at least a portion of the elongate shaft.

In Example 2, the subject matter of Example 1 optionally includes, wherein the one or more cameras comprise a Complementary Metal-Oxide Semiconductor Active Pixel Sensors (CMOS-APS).

In Example 3, the subject matter of Example 2 optionally includes, wherein one or more of the CMOS-APS are configured for sensing at an infrared wavelength range.

In Example 4, the subject matter of Examples 2-3 optionally includes, wherein the cannula is configured for a single use being constructed of a polymeric material and the pathway extends within the polymeric material.

In Example 5, the subject matter of Examples 1-4 optionally includes, wherein the elongate shaft has a ribbed outer surface along at least a portion thereof.

In Example 6, the subject matter of Examples 1-5 optionally includes, wherein an intermediate portion of the elongate shaft is constructed of a flexible material configured to allow the distal end portion of the elongate shaft to move from a first position to a second position.

In Example 7, the subject matter of Example 6 optionally includes, a mechanism to drive the distal end portion to articulate from the first position to the second position.

In Example 8, the subject matter of Examples 1-7 optionally includes, wherein the visualization assembly includes a light source comprising one or more light emitting diodes, and wherein the one or more cameras and the one or more light emitting diodes are positioned at the distal end portion.

In Example 9, the subject matter of Examples 1-8 optionally includes, wherein the visualization assembly is configured to be at least one of: advanced, retracted and rotated relative to the distal end portion of the elongate shaft.

In Example 10, the subject matter of Examples 1-9 optionally includes, a second cannula having a second visualization assembly including one or more cameras; a display apparatus; and a controller electronically coupled to the display apparatus, the visualization assembly and the second visualization assembly, wherein the controller is configured control the visualization assembly and the second visualization assembly and the display apparatus to cause the display apparatus to at least one of: change between a first field of view of the visualization assembly and a second field of view of the second visualization assembly, provide both the first field of view and the second field of view simultaneously or provide a three-dimensional composite view based upon the first field of view and the second field of view sharing substantially a same focal point.

In Example 11, the subject matter of Example 10 optionally includes, wherein the change between the first field of view and the second field of view is determined by the controller based upon an operation mode of the arthroscopic or laparoscopic device.

In Example 12, the subject matter of Examples 10-11 optionally includes, wherein the visualization assembly includes a first one or more light sources and the second visualization assembly includes a second one or more light sources, wherein the controller is configured to control the first one or more light sources and the second one or more light sources to be one or more of: activated to produce light at a same time; activated in sequence over a period of time; activated to produce light at the same time but with the first one or more light sources having a brightness that differs from a brightness of the second one or more light sources.

In Example 13, the subject matter of Examples 10-12 optionally includes, wherein the controller is configured to control articulation of the distal end portion relative to the proximal end portion of the elongate shaft to change the first field of view of the visualization assembly.

In Example 14, the subject matter of Examples 10-13 optionally includes, wherein the controller is configured to control the visualization assembly to at least one of advance, retract and rotate relative to the distal end portion of the elongate shaft.

Example 15 is a surgical system optionally comprising: an arthroscopic or laparoscopic device; a cannula having an elongate shaft configured to provide an access port to a target anatomical location within a patient for the arthroscopic or laparoscopic device, wherein the cannula includes, a visualization assembly having one or more cameras, the one or more cameras coupled to the elongate shaft of the cannula; and a controller electronically coupled to the visualization assembly, wherein the controller is configured control the visualization assembly to cause the visualization assembly to at least one of advance, retract and rotate relative to a distal end portion of the elongate shaft to alter a field of view of the one or more cameras.

In Example 16, the subject matter of Example 15 optionally includes, wherein the one or more cameras comprise a Complementary Metal-Oxide Semiconductor Active Pixel Sensors (CMOS-APS).

In Example 17, the subject matter of Example 16 optionally includes, wherein one or more of the CMOS-APS are configured for sensing at an infrared wavelength range.

In Example 18, the subject matter of Examples 16-17 optionally includes, wherein the cannula is configured for a single use being constructed of a polymeric material.

In Example 19, the subject matter of Examples 15-18 optionally includes, wherein the elongate shaft has a ribbed outer surface along at least a portion thereof.

In Example 20, the subject matter of Examples 15-19 optionally includes, wherein an intermediate portion of the elongate shaft is constructed of a flexible material configured to allow the distal end portion of the elongate shaft to move from a first position to a second position.

In Example 21, the subject matter of Example 20 optionally includes, a mechanism to drive the distal end portion to articulate from the first position to the second position.

In Example 22, the subject matter of Examples 15-21 includes, wherein the visualization assembly includes a light source comprising one or more light emitting diodes, and wherein the one or more cameras and the one or more light emitting diodes are positioned at the distal end portion.

In Example 23, the subject matter of Examples 15-22 optionally includes, wherein the visualization assembly is configured to be at least one of: advanced, retracted and rotated relative to the distal end portion of the elongate shaft.

In Example 24, the subject matter of Examples 15-23 optionally includes, a second cannula having a second visualization assembly including one or more cameras; and a display apparatus; wherein the controller electronically coupled to the display apparatus, the visualization assembly and the second visualization assembly, wherein the controller is configured control the visualization assembly and the second visualization assembly and the display apparatus to cause the display apparatus to at least one of: change between a first field of view of the visualization assembly and a second field of view of the second visualization assembly, provide both the first field of view and the second field of view simultaneously or provide a three-dimensional composite view based upon the first field of view and the second field of view sharing substantially a same focal point.

In Example 25, the subject matter of Example 24 optionally includes, wherein the change between the first field of view and the second field of view is determined by the controller based upon an operation mode of the arthroscopic or laparoscopic device.

In Example 26, the subject matter of Examples 24-25 optionally includes, wherein the visualization assembly includes a first one or more light sources and the second visualization assembly includes a second one or more light sources, wherein the controller is configured to control the first one or more light sources and the second one or more light sources to be one or more of: activated to produce light at a same time; activated in sequence over a period of time; activated to produce light at the same time but with the first one or more light sources having a brightness that differs from a brightness of the second one or more light sources.

In Example 27, the subject matter of Examples 15-26 optionally includes, wherein the controller is configured to control articulation of the distal end portion of the elongate shaft to change the field of view of the visualization assembly.

Example 28 is a surgical system optionally comprising: an arthroscopic or laparoscopic device; a cannula having an elongate shaft configured to provide an access port to a target anatomical location within a patient for the arthroscopic or laparoscopic device, wherein the cannula includes, a visualization assembly having one or more cameras, the one or more cameras coupled to the elongate shaft of the cannula; and a controller electronically coupled to the visualization assembly, wherein the controller is configured control articulation of a distal end portion of the elongate shaft relative to a proximal end portion of the elongate shaft to change a first field of view of the visualization assembly.

In Example 29, the subject matter of Example 28 optionally includes, wherein the one or more cameras comprise a Complementary Metal-Oxide Semiconductor Active Pixel Sensors (CMOS-APS).

In Example 30, the subject matter of Example 29 optionally includes, wherein one or more of the CMOS-APS are configured for sensing at an infrared wavelength range.

In Example 31, the subject matter of Examples 29-30 optionally includes, wherein the cannula is configured for a single use being constructed of a polymeric material.

In Example 32, the subject matter of Examples 28-31 optionally includes, wherein the elongate shaft has a ribbed outer surface along at least a portion thereof.

In Example 33, the subject matter of Examples 28-32 optionally includes, wherein an intermediate portion of the elongate shaft is constructed of a flexible material configured to allow the distal end portion of the elongate shaft to move from a first position to a second position.

In Example 34, the subject matter of Example 33 optionally includes, a mechanism to drive the distal end portion to articulate from the first position to the second position.

In Example 35, the subject matter of Examples 28-34 optionally includes, wherein the visualization assembly includes a light source comprising one or more light emitting diodes, and wherein the one or more cameras and the one or more light emitting diodes are positioned at the distal end portion.

In Example 36, the subject matter of Examples 28-35 optionally includes, wherein the visualization assembly is configured to be at least one of: advanced, retracted and rotated relative to the distal end portion of the elongate shaft.

In Example 37, the subject matter of Examples 28-36 optionally includes, a second cannula having a second visualization assembly including one or more cameras; and a display apparatus; wherein the controller electronically coupled to the display apparatus, the visualization assembly and the second visualization assembly, wherein the controller is configured control the visualization assembly and the second visualization assembly and the display apparatus to cause the display apparatus to at least one of: change between a first field of view of the visualization assembly and a second field of view of the second visualization assembly, provide both the first field of view and the second field of view simultaneously or provide a three-dimensional composite view based upon the first field of view and the second field of view sharing substantially a same focal point.

In Example 38, the subject matter of Example 37 optionally includes, wherein the change between the first field of view and the second field of view is determined by the controller based upon an operation mode of the arthroscopic or laparoscopic device.

In Example 39, the subject matter of Examples 37-38 optionally includes, wherein the visualization assembly includes a first one or more light sources and the second visualization assembly includes a second one or more light sources, wherein the controller is configured to control the first one or more light sources and the second one or more light sources to be one or more of: activated to produce light at a same time; activated in sequence over a period of time; activated to produce light at the same time but with the first one or more light sources having a brightness that differs from a brightness of the second one or more light sources.

In Example 40, the subject matter of Examples 28-39 optionally includes, wherein the controller is configured to control the visualization assembly to at least one of advance, retract and rotate relative to the distal end portion of the elongate shaft.

In Example 41, the subject matter of Examples 10-40 optionally includes, wherein the one or more cameras include an infrared camera, and wherein using data sensed by the infrared camera, the controller is configured to identify at least one of: a change in temperature in the joint space or a presence of excessive bleeding in the joint space.

Example 42 is at least one machine-readable medium including instructions that, when executed by processing circuitry, cause the processing circuitry to perform operations to implement of any of Examples 1-41.

Example 43 is an apparatus comprising means to implement of any of Examples 1-41.

Example 44 is a system to implement of any of Examples 1-41.

Example 45 is a method to implement of any of Examples 1-41.

In Example 46, the devices, methods or systems of any one or any combination of Examples 1-45 can optionally be configured such that all elements or options recited are available to use or select from.

### BRIEF DESCRIPTION OF THE DRAWINGS

Various embodiments of the present invention will now be discussed with reference to the appended drawings. It should be appreciated that the drawings depict only typical embodiments of the invention and are therefore not to be considered limiting in scope.

FIG. 1 is a schematic view of an arthroscopic cannula that includes a visualization assembly according to an example of the present disclosure.

FIG. 2 is a schematic view of another arthroscopic cannula that includes a second visualization assembly with the second visualization assembly configured to be moveable according to an example of the present disclosure.

FIG. 3 is a schematic view of yet another arthroscopic cannula that includes a third visualization assembly; the arthroscopic cannula configured to have a moveable distal end according to an example of the present disclosure.

FIG. 4A is a side view of an arthroscopic cannula with a fourth visualization assembly that includes two integrated cameras of different types according to an example of the present disclosure.

FIG. 4B is a side view from a distal side of the arthroscopic cannula and the fourth visualization assembly of FIG. 4A.

FIG. 5 shows the arthroscopic cannula of FIG. 3 being inserted into a hip joint of a patient according to an example of the present disclosure.

FIG. 6 shows a view of an interior of the hip joint with a cutting instrument performing removal of tissue.

FIG. 7 shows a view of an interior of a shoulder joint obtained using one of the arthroscopic cannulas of FIGS. 1-4B.

FIG. 8 is a schematic view of a control unit controlling various operations of an electrosurgical device and an arthroscopic cannula according to an example of the present disclosure.

FIGS. 9-11 schematically illustrate a controller controlling various display functions on a display using imaging from one or both of first visualization assembly of a first arthroscopic cannula and the second visualization assembly of a second arthroscopic cannula.

FIG. 12 illustrates a block diagram of an example machine upon which any one or more of the techniques, apparatuses, systems or methods discussed herein may perform in accordance with at least one example of this disclosure.

### DETAILED DESCRIPTION OF THE INVENTION

The present invention relates to an arthroscopic cannula with a visualization assembly that can include integrated camera(s) chip and LED light source(s). Several embodiments of the arthroscopic cannula will now be described to provide an overall understanding of the principles of the form, function and methods of use. The arthroscopic cannulas can be used with other arthroscopic tools that provide therapy such as mechanical or radiofrequency (RF) cutting of tissue. The arthroscopic cannulas can be disposable. However, such devices can be reusable according to some examples. This description of the general principles of this invention is not meant to limit the inventive concepts in the appended claims.

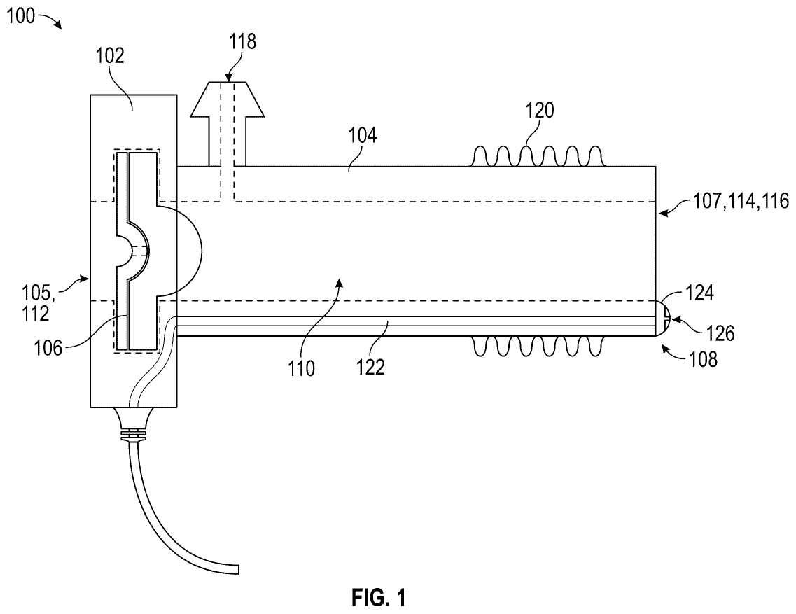

FIG. 1 shows an arthroscopic cannula 100 according to one example. The arthroscopic cannula 100 can include a main body 102, an elongate shaft 104, a seal(s) 106 and a visualization assembly 108. The elongate shaft 104 can include a lumen(s) 110, a first port 112, a second port 114, a distal tip 116, a third port 118 and a ribbed portion 120. The visualization assembly 108 can include cable(s) 122, one or more cameras 124 and one or more light sources 126.

The elongate shaft 104 can have a proximal end portion 105 and an opposing distal end portion 107. The proximal end portion 105 can be connected to the main body 102. The main body 102 and the elongate shaft 104 can be constructed of suitable rigid or semi-rigid biocompatible material(s) such as polymeric material(s). Suitable polymeric material(s) can include polycarbonate. The suitable material(s) can optionally be transparent for visualizing instrument(s) passing through the arthroscopic cannula 100. The arthroscopic cannula 100 can be disposable after a single use, for example. The main body 102 can be integrally formed with elongate shaft 104 so as to be a single piece construct or can be connected thereto by suitable mechanical coupling. The seal(s) 106 can be a trocar seal placed at the proximal end portion 105 of the elongate shaft 104 and/or within the main body 102. The seal(s) 106 can be positioned adjacent the first port 112, for example.

The lumen(s) 110 can extend through the elongate shaft 104 and the main body 102 and can be in communication with the first port 112 and the second port 114. The first port 112 can be on a proximal end of the arthroscopic cannula 100 while the second port 114 can be on a distal end of the arthroscopic cannula 100 at the distal tip 116. The third port 118 can communicate with the lumen(s) 110 through a side passage in the elongate shaft 104 and/or the main body 102. The ribbed portion 120 can be part of the elongate shaft 104 and can be positioned proximal of the distal tip 116. The ribbed portion 120 can be located along an outer surface of the elongate shaft 104 and can project radially outward of a main surface of the elongate shaft 104.

The visualization assembly 108 can be coupled to the elongate shaft 104 and/or the main body 102. The one or more cameras 124 and/or the one or more light sources 126 can be located at or adjacent the distal tip 116 such as adjacent or within the second port 114. The visualization assembly 108 can include components such as parts of the cable(s) 122 that are integrated (formed within or pass through) the main body 102 and/or the elongate shaft 104. Thus, the visualization assembly 108 can have a pathway (e.g., cable(s) 122) for optical and electrical connection to the one or more cameras 124 and/or the one or more light sources 126 with the pathway formed along at least a portion of the elongate shaft 104.

The elongate shaft 104 is insertable to extend within a desired anatomical space of the patient. The seal(s) 106 can comprise a trocar or other type seal that can selectively close access between the first port 112 and portions of the lumen(s) 110. The lumen(s) 110 can provide a working channel for an arthroscopic or laparoscopic device to pass through to the distal end portion 107 of the elongate shaft 104. The diameter of the working channel can between 4 mm and 12 mm, for example. The third port 118 can communicate with the lumen(s) to provide a port for fluid (e.g., saline) inflow to the working channel, for example. The ribbed portion 120 on an exterior of the elongate shaft 104 can be configured to increase friction with the dermal and muscle tissue the elongate shaft 104 passes through.

The cable(s) 122 of the visualization assembly 108 can pass though the main body 102 and/or the elongate shaft 104 to the distal tip 116. The one or more light sources 126 can include one or more light emitting diodes (LEDs) or other illumination components. The LED can be an on-chip controlled devices with the chip being at or adjacent the distal tip 116 of the elongate shaft 104. However, other locations for the chip and/or the one or more light sources 126 on the elongate shaft 104 proximal of the distal tip 116 are contemplated. The one or more cameras 124 can be an on-chip controlled devices with the chip being at or adjacent the distal tip 116 of the elongate shaft 104. However, other locations for the chip and/or the one or more cameras 124 on the elongate shaft 104 proximal of the distal tip 116 are contemplated. For example, the one or more cameras 124 can utilize Complementary Metal-Oxide Semiconductor Active Pixel Sensors (CMOS-APS). The CMOS-APS are configured for sensing at an infrared wavelength range, a visual light wavelength range or another wavelength range as desired.

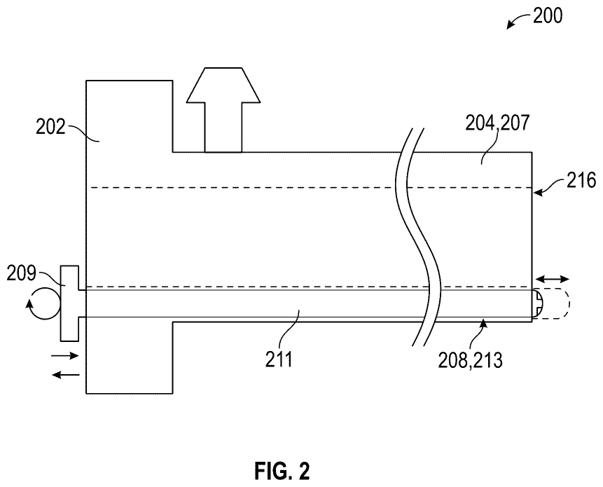

FIG. 2 shows another example of an arthroscopic cannula 200. The arthroscopic cannula 200 can have a construction similar to that of the arthroscopic cannula 100 of FIG. 1. However, the arthroscopic cannula 200 include a visualization assembly 208 that is moveable relative to a main body 202 and/or an elongate shaft 204. In particular, the visualization assembly 208 can include an actuator 209 and a shaft 211. The shaft 211 can be coupled to the one or more cameras 124 and/or the one or more light sources 126. The shaft 211 can extend within a channel 213 of the elongate shaft 204 and is moveable within such channel 213 such that the shaft 211 is independently moveable relative to the elongate shaft 204. The channel 213 can be separate from the lumen(s) 110. The shaft 211 can be rigid or semi-rigid and can include a pathway (e.g., cable(s) 122) for optical and electrical connection to the one or more cameras 124 and/or the one or more light sources 126.

The shaft 211 can be integrally formed with or otherwise mechanically coupled to the actuator 209. The actuator 209 can be proximal of the main body 202 and/or the elongate shaft 204. The actuator 209 can be graspable or otherwise manipulatable by a surgeon or a robotic device connected to a controller (see subsequent discussion).

As illustrated in FIG. 2, the actuator 209 and/or the shaft 211 allow the one or more cameras 124 and/or the one or more light sources 126 to be moveable relative to the elongate shaft 204, in particular, a distal tip 216 of the elongate shaft 204. Thus, as shown in FIG. 2, the visualization assembly 208, in particular the one or more cameras 124 and/or the one or more light sources 126 via the actuator 209 and the shaft 211, can be advanced, retracted and rotated in position relative to the distal tip 216 and a distal end portion 207 of the elongate shaft 204. Thus, the visualization assembly 208 can be advanced linearly, retracted linearly, and/or rotated as desired. This moveability can alter a field of view of the one or more cameras 124, for example.

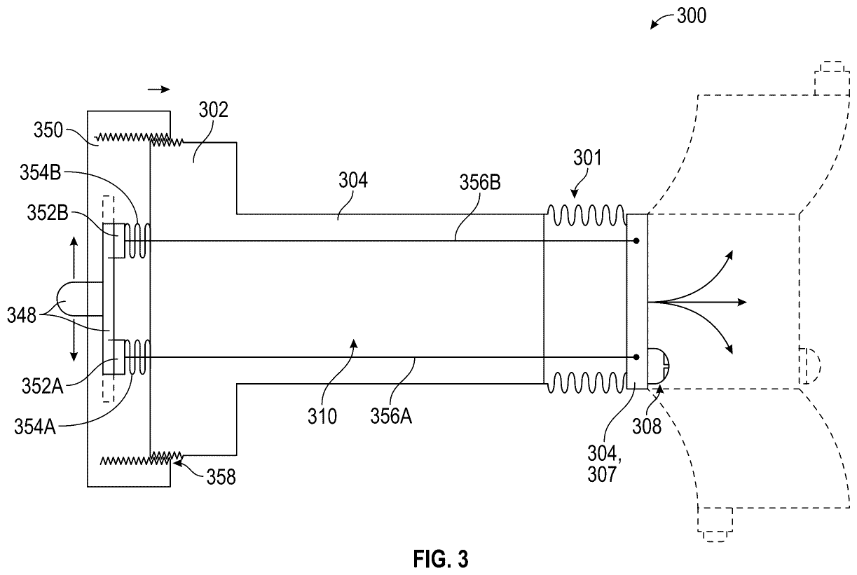

FIG. 3 shows another example of an arthroscopic cannula 300. The arthroscopic cannula 300 has an advanceable and/or articulating distal end portion 307 of an elongate shaft 304 and a visualization assembly 308. This configuration allows for the manipulation of the distal end portion 307 within the anatomy (e.g., a joint). This manipulation of the distal end portion 307 can allow the arthroscopic cannula 300 to be steered in the manner of an endoscope. This allows a lumen(s) 310 defined by the elongate shaft 304 to be steered toward target tissue. According to one example, steering of the arthroscopic cannula 300 can be performed by a controller (see discussion below) controlling a robotic device and can be automated to visually follow the surgical instrument placed through the lumen(s) 310 of the elongate shaft 304.

As shown in FIG. 3, an intermediate portion 301 of the elongate shaft 304 is constructed of a flexible material (e.g., silicone) such as a membrane. The flexible material can be configured to allow the distal end portion 307 of the elongate shaft 304 to move from a first position to a second position (both shown in phantom in FIG. 3).

A mechanism 303 can be used to drive the distal end portion 307 of the elongate shaft 304 to articulate to the first position or the second position. According to one example, the mechanism 303 can include an actuator 348, a rotating cap 350, a plurality of collars 352A and 352B, one or more springs 354A and 354B and a plurality of guide wires 356A and 356B. The mechanism 303 can be located at a proximal end portion such as at or adjacent a main body 302. The actuator 348 can be captured by and can be movable laterally within the rotating cap 350. The actuator 348 can be a lever according to one example. In a neutral position as shown in FIG. 3, the plurality of collars 352A and 352B can both be in selective contact with the actuator 348 as forced by respective ones of the one or more springs 354A and 354B. The rotating cap 350 can be moveably coupled to the main body 302 or the elongate shaft 304 by threads 358 or other mechanical mechanism that allows for relative controlled movement between components. The rotating cap 350 can receive the plurality of collars 352A and 352B. The plurality of guide wires 356A and 356B can be coupled to respective ones of the plurality of collars 352A and 352B by welding or other joining technique. The one or more springs 354A and 354B can be positioned between the rotating cap 350 and the main body 302 and/or the elongate shaft 304. The plurality of guide wires 356A and 356B can pass through the intermediate portion 301 and can be connected to a part of the elongate shaft 304 distal of the intermediate portion 301. The plurality of guide wires 356A and 356B can comprise rigid or semi-rigid shafts, for example.

Rotating the rotating cap 350 when the actuator 348 is in the neutral position presses down on both the plurality of guide wires 356A and 356B coupled to the plurality of collars 352A and 352B. As the rotating cap 350 is rotated to the viewer's right, the plurality of guide wires 356A and 356B are advanced, which extends the distal end portion 307 by expansion of the intermediate portion 301 (e.g., a flexible membrane). Such extension of the distal end portion 307 is indicated in phantom in FIG. 3.

Additionally if bending of the intermediate portion 301 in a first direction is desired, the actuator 348 can be moved laterally (e.g., in a first direction up or down to the viewer as indicated in phantom). This lateral movement can bring the actuator 348 out of contact with a first of the plurality of collars 352A, while contact is maintained between the actuator 348 and a second of the plurality of collars 352B. A first of the plurality of guide wires 356A coupled to the non-contacting first of the plurality of collars 352A is disconnected from the actuator 348 (not in contact therewith via the first plurality of collars 352A) such that as the rotating cap 350 is rotated to the viewer's right, the first of the plurality of guide wires 356A does not advance (only the second of the plurality of guide wires 356B, which in contact with the actuator 348 via the second of the plurality of collars 352B advances). This advancement causes the bending of the intermediate portion 301 and the distal end portion 307 to the first position shown in phantom in FIG. 3.

If bending of the intermediate portion 301 in a second direction is desired, the actuator 348 can be moved laterally (e.g., in a second direction as indicated up or down to the viewer in phantom). This lateral movement can bring the actuator 348 out of contact with the second of the plurality of collars 352B, while contact is maintained between the actuator 348 and the first of the plurality of collars 352A. The second of the plurality of guide wires 356B coupled to the non-contacting second of the plurality of collars 352B is disconnected from the actuator 348 (as the second of the plurality of collars 352B are not in contact therewith) such that as the rotating cap 350 is rotated to the viewer's right, the second of the plurality of guide wires 356B does not advance (only the first of the plurality of guide wires 356A in contact with the actuator 348 advances). This advancement causes the bending of the intermediate portion 301 and the distal end portion 307 to the second position shown in phantom in FIG. 3.

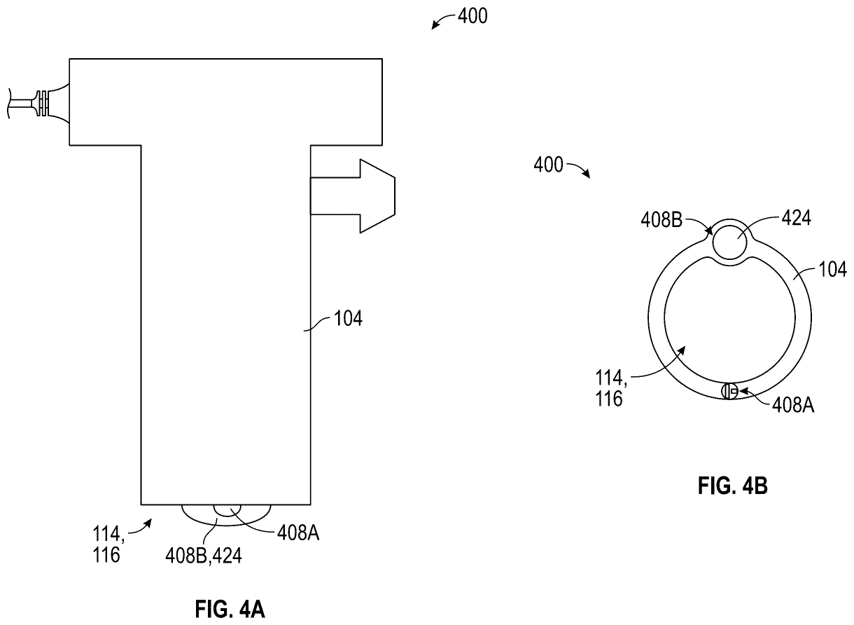

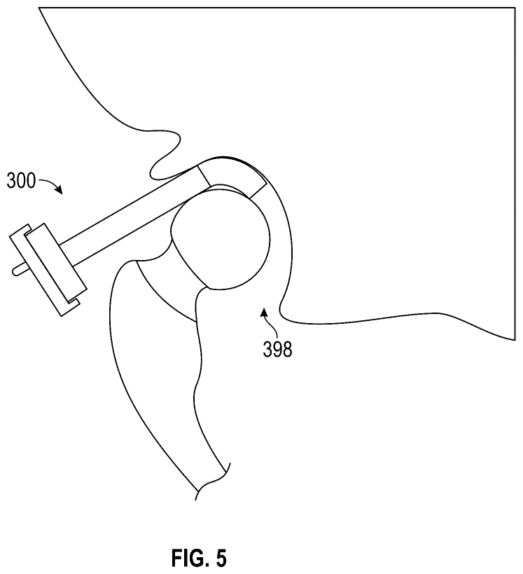

FIGS. 4A and 4B show another example of an arthroscopic cannula 400. The arthroscopic cannula 400 can have a construction similar to that of the arthroscopic cannula 100 of FIG. 1. However, the arthroscopic cannula 400 include a first visualization assembly 408A and a second visualization assembly 408B. The first visualization assembly 408A can be constructed in the manner of the visualization assembly 108 of FIG. 1. The second visualization assembly 408B can include an infrared camera 424 configured for sensing at an infrared wavelength range. The second visualization assembly 408B can be positioned across the second port 114 of the elongate shaft 104 from the first visualization assembly 408A at the distal tip 116 of the elongate shaft 104. The infrared camera 424, in concert with the controller (see discussion below), can be configured to monitor the joint space for temperature changes caused by radiofrequency devices. An elevated temperature(s) sensed using the infrared camera 424 can be electronically communicated to the controller. Based upon such sensed elevated temperature(s), the controller can increase fluid flow to the joint, for example. Additionally, or alternatively, the infrared camera 424 can also be used to detect blood such as excessive bleeding by using an algorithm(s) of the controller. Such algorithm(s) run on the controller can, for example, be used to identify a temperature difference between the arthroscopic fluid (e.g., saline) and the blood (e.g., about 37 degrees) that can seep from the joint. This difference can be sensed using the infrared camera 424 and identified with suitable algorithm(s). In some applications, the infrared camera 424 can be used for early detection of bleeding in microfracture procedures such that techniques discussed above are not limited to total or partial joint replacement procedures.

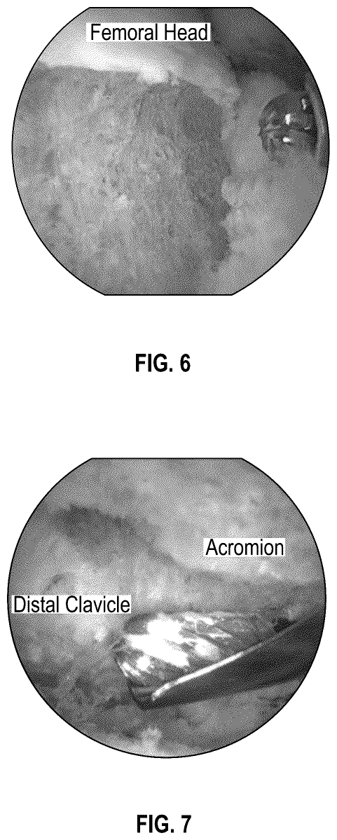

FIG. 5 illustrates use of the arthroscopic cannula 300 within a hip joint 398. The arthroscopic cannula 300 enables the ability to perform a hip arthroscopy though a single access port (using the single arthroscopic cannula 300). The arthroscopic cannula 300 functions both as the traditional endoscope as well as a “sled”, which is used to introduce therapeutic device(s) into the joint. The articulated end of the arthroscopic cannula 300 will guide the therapeutic device(s) to the treatment area.

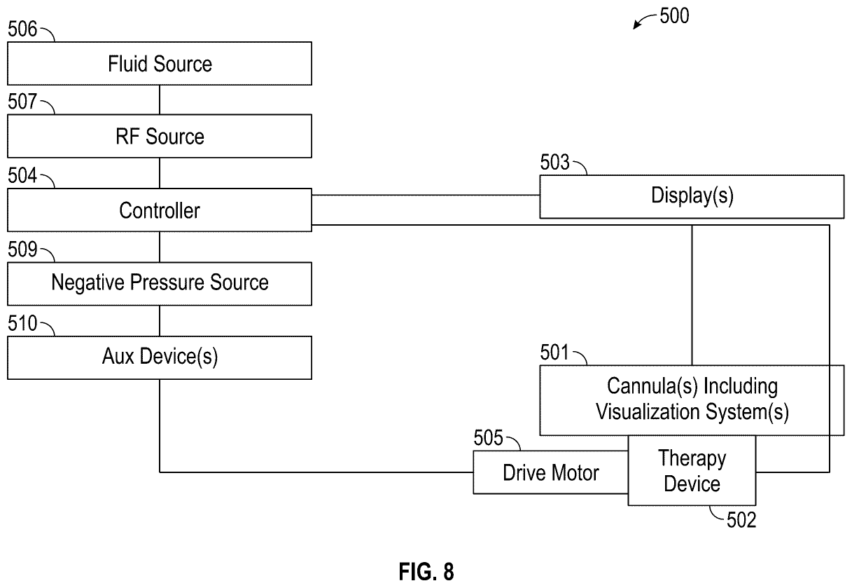

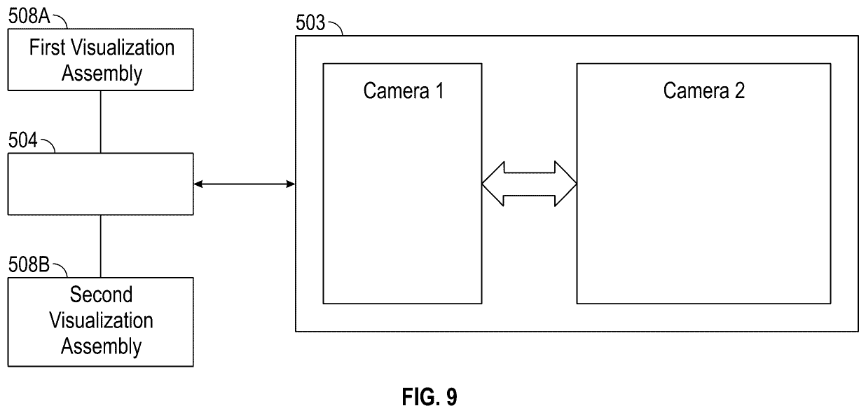

FIGS. 6 and 7 show therapeutic instruments (here burrs) being used to remove tissue within the hip joint (FIG. 6) and the shoulder joint (FIG. 7). The images of FIGS. 6 and 7 were captured using one of the arthroscopic cannulas discussed herein.

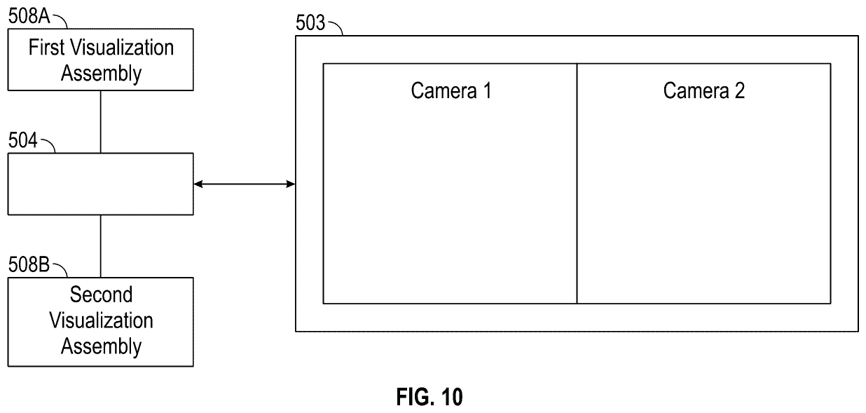

FIG. 8 shows an arthroscopic system 500 including an arthroscopic cannula 601, a therapeutic device 502, a display 503 and a controller 504. The therapeutic device 502 can be an electrosurgical apparatus described in the various applications incorporated by reference with the U.S. Application Publications noted above. The therapeutic device 502 can be a probe that has a distal (working) end that carries tissue cutting mechanism(s) optionally driven by a drive motor 505, and RF electrode(s) coupled to an RF source 507. The RF electrode(s) of the probe can be configured for use in many arthroscopic surgical applications, including but not limited to treating bone in shoulders, knees, hips, wrists, ankles and the spine.

Controller 504 can be configured to control various aspects of the arthroscopic system 500 including the RF energy provided to the electrode(s), control the visualization assembly(s) for illumination and/or image capture, control fluid inflow from fluid source 506 and/or fluid removal to negative pressure source 509, can control operation of the drive motor 605 for mechanical cutting or the like.

In FIG. 8, it can be seen that the controller 504 is operatively electronically coupled the arthroscopic cannula 501, the therapeutic device 502, and the display 503. Additionally, the controller 504 which can be operatively electronically coupled to control the drive motor 505, control communication with a fluid source 506, control communication with the RF source 507, control communication with the negative pressure source 509 and can control various auxiliary devices 510 (e.g., a robotic surgical device). The display 503 can provided for displaying operating parameters, images that are captured by the visualization assembly(s), etc.

The system 500 can include a flow inducing device (not shown) such as a pump, positive pressure source or the like that is in fluid communication from the fluid source 506. The flow inducing device can allow for fluid flow to the anatomy being operated upon of an irrigating fluid (e.g., saline) utilized during operation of the therapeutic device 502. As can be understood from the above description of the system 500, the therapeutic device 502 and the arthroscopic cannula 501, the controller 504 and controller algorithms can be configured to perform and automate many tasks to provide for system functionality.

The controller 504 can include, for example, software, hardware, and combinations of hardware and software configured to execute several functions related to, among others, operation of the system 500 (FIG. 8). The controller 504 can be an analog, digital, or combination analog and digital controller including a number of components. As examples, the controller 504 can include integrated circuit boards or ICB(s), printed circuit boards PCB(s), processor(s), data storage devices, switches, relays, or any other components. Examples of processors can include any one or more of a microprocessor, a controller, a digital signal processor (DSP), an application specific integrated circuit (ASIC), a field-programmable gate array (FPGA), or equivalent discrete or integrated logic circuitry. Commercially available microprocessors can be configured to perform the functions of the controller 504. Various known circuits may be associated with controller 504, including power supply circuitry, signal-conditioning circuitry, actuator driver circuitry (i.e., circuitry powering solenoids, motors, or piezo actuators), and communication circuitry. In some examples, the controller 504 may be part of a control unit.

The controller 504 can include a memory such as memory circuitry. The memory may include storage media to store and/or retrieve data or other information such as, operational algorithms. Storage devices, in some examples can be a computer-readable storage medium. The data storage devices can be used to store program instructions for execution by processor(s) of the controller 504, for example. The storage devices, for example, are used by software, applications, algorithms, as examples, running on and/or executed by the controller 504. The storage devices can include short-term and/or long-term memory and can be volatile and/or non-volatile. Examples of non-volatile storage elements include magnetic hard discs, optical discs, floppy discs, flash memories, or forms of electrically programmable memories (EPROM) or electrically erasable and programmable (EEPROM) memories. Examples of volatile memories include random access memories (RAM), dynamic random-access memories (DRAM), static random-access memories (SRAM), and other forms of volatile memories known in the art.

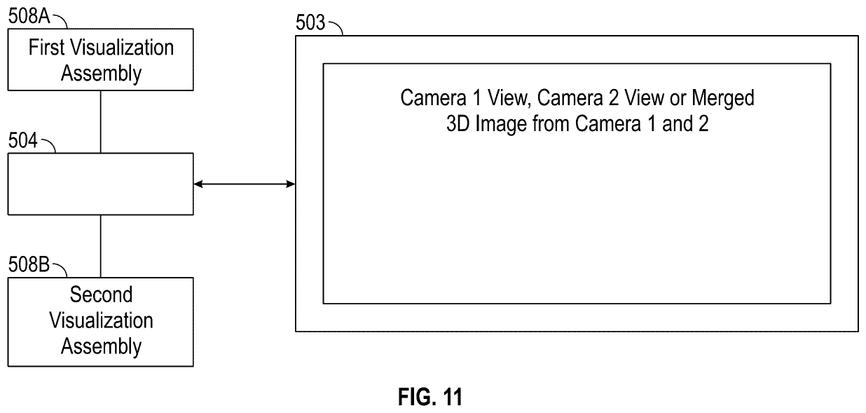

FIGS. 9-11 show applications of aspects of the system 500 including the controller 504 and display 503 along with a first visualization assembly 508A and a second visualization assembly 508B. The first visualization assembly 508A can be from a first device such as the arthroscopic cannula 501 (FIG. 8). The second visualization assembly 508B can be from a second arthroscopic cannula used during surgery, the first device, or from the therapeutic device 502, for example. The controller 504 can be configured control the first visualization assembly 508A and the second visualization assembly 508B and the display 503 to cause the display 503 to perform various functions.

The first visualization assembly 508A and the second visualization assembly 508B can include a light source and/or one or more cameras as previously discussed. According to some examples the light sources of the first visualization assembly 508A and the second visualization assembly 508B can be identical or can differ from one another (e.g., one light source can be configured for colored illumination while the other light source can be configured for white light illumination, the one light source can be brighter and/or differently arranged then the other light source, etc.). Similarly, the one or more cameras of the first visualization assembly 508A and the second visualization assembly 508B can be identical or can differ from one another (e.g., at least one of the one or more cameras can be configured for infrared sensing while others of the one or more cameras can be configured for visual light sensing, etc.).

The light sources can be selectively controlled. Thus, light sources can be activated at a same time, can be activated separately, a brightness of each of the light sources can be controlled independently, etc. The light sources can be configured to selectively increase or decrease in luminance, change illumination color, etc. under control. The light sources can be activated in sequence over a period of time. The light sources can be activated to produce light at the same time but with the light sources can have a brightness that differs from one another.

The one or more cameras of the first visualization assembly 508A and the one or more cameras of the second visualization assembly 508B can be positioned so as to have field of views that includes different parts of the therapeutic device and/or tissue. The one or more cameras of the first visualization assembly 508A and the one or more cameras of the second visualization assembly 508B can be selectively controlled. According to one example, the one or more cameras of the first visualization assembly 508A and the one or more cameras of the second visualization assembly 508B can be active/operational at the same time and can have the same focal point. Because the one or more cameras of the first visualization assembly 508A and the one or more cameras of the second visualization assembly 508B are at different distances or orientations but can share the same focal point a 3D composite image can be captured for processing.

FIGS. 9-11 show the controller 504 can be configured control the first visualization assembly 508A and the second visualization assembly 508B and the display 503 to cause the display 503 to at least one of: change between the first field of view first visualization assembly 508A and the second field of view second visualization assembly 508B (as shown in FIG. 15), provide both the first field of view and the second field of view simultaneously (as shown in FIG. 16) or provide a three-dimensional composite view based upon the first field of view and the second field of view sharing substantially a same focal point (example of FIG. 17). Thus, the controller 504 can be configured to toggle between the two or more camera views of the first visualization assembly 508A and the second visualization assembly 508B and/or the controller 504 can be configured to provide both camera views from the first visualization assembly 508A and the second visualization assembly 508B on the display 503 at the same time.

Alternatively, FIG. 15 or 17 illustrates that the change between the first field of view of the first visualization assembly 508A and the second field of view of the second visualization assembly 508B can be determined by the controller 504 based upon an operation mode of the medical device (the arthroscopic or laparoscopic device). In particular, the controller 504 can automatically switch between camera views from the first visualization assembly 508A and the second visualization assembly 508B based on device function. For example, during the diagnostic phase of the procedure the second visualization assembly 508B can be used if directed to view the tissue to be operated on and during activation phase (e.g., mechanical cutting, coagulation, ablation etc.) the camera view can automatically switch to the first visualization assembly 508A, which has a field of view that includes an end effector of the therapeutic device such that operation of the end effector can be monitored.

According to further examples, the controller 504 can be configured to control the first visualization assembly 508A and the second visualization assembly 508B to activate light sources to produce light at a same time, activate light sources in sequence over a period of time and/or activate light sources to produce light at the same time but with the first one or more light sources having a brightness that differs from a brightness of the second one or more light sources. The controller 508 via a robotic device can be configured to control articulation of the distal end portion relative to the proximal end portion of the elongate shaft to change the first field of view of the visualization assembly (automate and control the example shown in FIG. 3). The controller 504 via a robotic device can be configured to control the visualization assembly to at least one of advance, retract and rotate relative to the distal end portion of the elongate shaft (automate and control the example shown in FIG. 2).

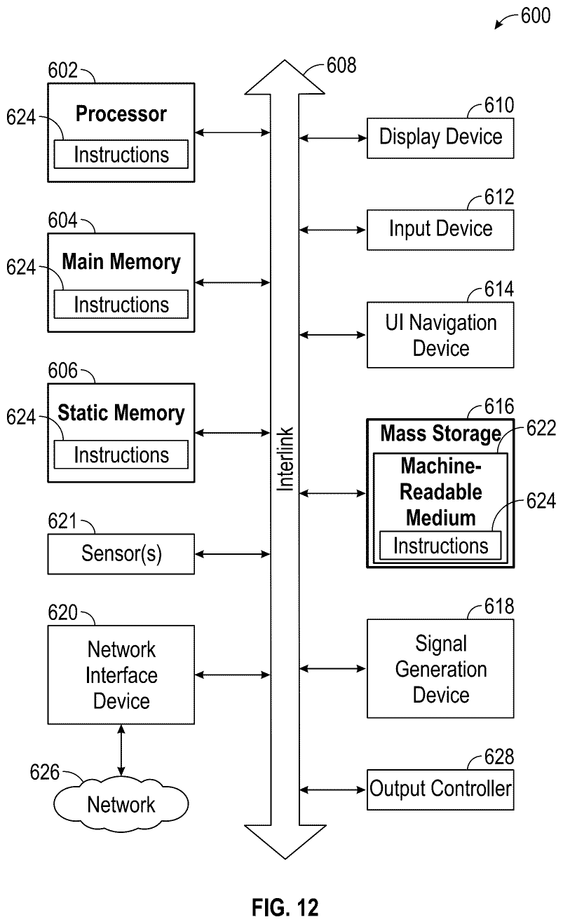

FIG. 12 illustrates a block diagram of an example machine 600 upon which any one or more of the techniques discussed herein may perform in accordance with some embodiments. This example machine can operate some or all of the apparatus and/or system function discussed herein. In other examples, the example machine 600 is merely one of many such machines utilized. In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610, an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and plurality of sensors 621, such as any of those discussed previously (e.g., an IMU, a global positioning system (GPS) sensor, compass, accelerometer, or other sensor). The machine 600 may include an output controller 628, such as a serial (e.g., Universal Serial Bus (USB), parallel, or other wired or wireless (e.g., infrared (IR), near field communication (NFC), etc.) connection to communicate or control one or more peripheral devices (e.g., a printer, card reader, etc.).

The storage device 616 may include a machine readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine readable media.

While the machine readable medium 622 is illustrated as a single medium, the term “machine readable medium” may include a single medium or multiple media (e.g., a centralized or distributed database, and/or associated caches and servers) configured to store the one or more instructions 624. The term “machine readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples may include solid-state memories, and optical and magnetic media.

The instructions 624 may further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The term “substantially”, “generally” or “about” mean within 15% of the value provided. The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Method and system examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

All references, including publications, patent applications, and patents, cited herein are hereby incorporated by reference to the same extent as if each reference were individually and specifically indicated to be incorporated by reference and were set forth in its entirety herein.

What is claimed is:

1. A cannula configured to provide an access port to a patient for an arthroscopic or laparoscopic device, comprising:
an elongate shaft having a proximal end portion and a distal end portion, wherein the elongate shaft includes one or more lumens therein including at least one lumen configured to provide a working channel for the arthroscopic or laparoscopic device to pass through the distal end portion;
a visualization assembly including one or more cameras, the visualization assembly coupled to the elongate shaft and having pathway for optical and electrical connection to the one or more cameras with the pathway formed along at least a portion of the elongate shaft.

2. The cannula of claim 1, wherein an intermediate portion of the elongate shaft is constructed of a flexible material configured to allow the distal end portion of the elongate shaft to move from a first position to a second position.

3. The cannula of claim 2, further comprising a mechanism to drive the distal end portion to articulate from the first position to the second position.

4. The cannula of claim 1, wherein the visualization assembly includes a light source comprising one or more light emitting diodes, and wherein the one or more cameras and the one or more light emitting diodes are positioned at the distal end portion.

5. The cannula of claim 1, wherein the visualization assembly is configured to be at least one of: advanced, retracted and rotated relative to the distal end portion of the elongate shaft, and wherein the one or more cameras comprise a Complementary Metal-Oxide Semiconductor Active Pixel Sensors (CMOS-APS) that are configured for sensing at an infrared wavelength range.

6. The cannula of claim 1, further comprising:
a second cannula having a second visualization assembly including one or more cameras;
a display apparatus; and
a controller electronically coupled to the display apparatus, the visualization assembly and the second visualization assembly, wherein the controller is configured control the visualization assembly and the second visualization assembly and the display apparatus to cause the display apparatus to at least one of: change between a first field of view of the visualization assembly and a second field of view of the second visualization assembly, provide both the first field of view and the second field of view simultaneously or provide a three-dimensional composite view based upon the first field of view and the second field of view sharing substantially a same focal point.

7. The cannula of claim 6, wherein the change between the first field of view and the second field of view is determined by the controller based upon an operation mode of the arthroscopic or laparoscopic device.

8. The cannula of claim 6, wherein the visualization assembly includes a first one or more light sources and the second visualization assembly includes a second one or more light sources, wherein the controller is configured to control the first one or more light sources and the second one or more light sources to be one or more of:
activated to produce light at a same time;
activated in sequence over a period of time;
activated to produce light at the same time but with the first one or more light sources having a brightness that differs from a brightness of the second one or more light sources.

9. The cannula of claim 8, wherein the controller is configured to control articulation of the distal end portion relative to the proximal end portion of the elongate shaft to change the first field of view of the visualization assembly.

10. The cannula of claim 8, wherein the controller is configured to control the visualization assembly to at least one of advance, retract and rotate relative to the distal end portion of the elongate shaft.

11. A surgical system comprising:
an arthroscopic or laparoscopic device;
a cannula having an elongate shaft configured to provide an access port to a target anatomical location within a patient for the arthroscopic or laparoscopic device, wherein the cannula includes a visualization assembly having one or more cameras, the one or more cameras coupled to the elongate shaft of the cannula; and
a controller electronically coupled to the visualization assembly, wherein the controller is configured control the visualization assembly to cause the visualization assembly to at least one of advance, retract and rotate relative to a distal end portion of the elongate shaft to alter a field of view of the one or more cameras.

12. The surgical system of claim 11, wherein the one or more cameras comprise a Complementary Metal-Oxide Semiconductor Active Pixel Sensors (CMOS-APS), wherein one or more of the CMOS-APS are configured for sensing at an infrared wavelength range.

13. The surgical system of claim 11, wherein the elongate shaft has a ribbed outer surface along at least a portion thereof.

14. The surgical system of claim 11, wherein an intermediate portion of the elongate shaft is constructed of a flexible material configured to allow the distal end portion of the elongate shaft to move from a first position to a second position.

15. The surgical system of claim 11, wherein the visualization assembly is configured to be at least one of: advanced, retracted and rotated relative to the distal end portion of the elongate shaft.

16. The surgical system of claim 11, further comprising:
a second cannula having a second visualization assembly including one or more cameras; and
a display apparatus;
wherein the controller electronically coupled to the display apparatus, the visualization assembly and the second visualization assembly, wherein the controller is configured control the visualization assembly and the second visualization assembly and the display apparatus to cause the display apparatus to at least one of:
change between a first field of view of the visualization assembly and a second field of view of the second visualization assembly, provide both the first field of view and the second field of view simultaneously or provide a three-dimensional composite view based upon the first field of view and the second field of view sharing substantially a same focal point.

17. The surgical system of claim 16, wherein the change between the first field of view and the second field of view is determined by the controller based upon an operation mode of the arthroscopic or laparoscopic device.

18. The surgical system of claim 17, wherein the visualization assembly includes a first one or more light sources and the second visualization assembly includes a second one or more light sources, wherein the controller is configured to control the first one or more light sources and the second one or more light sources to be one or more of:
activated to produce light at a same time;
activated in sequence over a period of time;
activated to produce light at the same time but with the first one or more light sources having a brightness that differs from a brightness of the second one or more light sources;
wherein the controller is configured to control articulation of the distal end portion of the elongate shaft to change the field of view of the visualization assembly.

19. A surgical system comprising:
an arthroscopic or laparoscopic device;
a cannula having an elongate shaft configured to provide an access port to a target anatomical location within a patient for the arthroscopic or laparoscopic device, wherein the cannula includes a visualization assembly having one or more cameras, the one or more cameras coupled to the elongate shaft of the cannula; and
a controller electronically coupled to the visualization assembly, wherein the controller is configured control articulation of a distal end portion of the elongate shaft relative to a proximal end portion of the elongate shaft to change a first field of view of the visualization assembly.

20. The cannula of claim 19, wherein the one or more cameras include an infrared camera, and wherein using data sensed by the infrared camera, the controller is configured to identify at least one of: a change in temperature in a joint space or a presence of excessive bleeding in the joint space.
